(19) United States
(12) Patent Application Publication
(10) Pub. No.: US 2019/0387980 A1
(43) Pub. Date: Dec. 26, 2019

(54) BLOOD PRESSURE MONITORING CUFF

(71) Applicant: SOCIONEXT INC., Yokohama (JP)

(72) Inventors: Hiroaki TAKAGI, Yokohama (JP); Masaya TAMAMURA, Yokohama (JP); Amane INOUE, Yokohama (JP); Minoru NAKAGAWARA, Sapporo (JP)

(21) Appl. No.: 16/563,583

(22) Filed: Sep. 6, 2019

Related U.S. Application Data
(63) Continuation of application No. PCT/JP2017/014786, filed on Apr. 11, 2017.

(51) Int. Cl. A61B 5/022; A61B 5/00

(52) U.S. Cl. CPC A61B 5/022; A61B 5/6831

(57) ABSTRACT

A cuff includes a band that is wound around a measuring position where blood pressure is measured and an air bag that is provided on a measuring position side of the band and whose length in a length direction is shorter than an outer circumference of the measuring position. A first spacer and a second spacer are respectively provided at the ends in the length direction of the air bag. The band is wound around the measuring position so as to cover the first spacer and the second spacer and detachably fixed by a fixing portion to a surface on an opposite side to the measuring position side.

### CROSS-REFERENCE TO RELATED APPLICATION

This application is a continuation application of International Application PCT/JP2017/014786 filed on Apr. 11, 2017 which designated the U.S., the entire contents of which are incorporated herein by reference.

### FIELD

The embodiments discussed herein are related to a blood pressure monitoring cuff.

### BACKGROUND

Several blood pressure monitoring cuffs, which measure blood pressure by being wound around a measuring position such as the arm, wrist, or finger, are known. Measurement of blood pressure using a cuff is performed by winding the cuff around a predetermined measuring position and pressurizing (i.e., inflating) an air bag (also referred to by terms such as “expansion chamber”) provided on the cuff to compress an artery at the measuring position.

See, for example, the following documents:

Japanese Laid-open Patent Publication No. 2003-144397; and

Japanese Laid-open Patent Publication No. S63-77433.

When blood pressure is measured using a cuff, pressurizing the cuff in a state where the cuff surrounds the entire circumference of the measuring position compresses veins and may cause congestion. Congestion may cause discomfort to the subject and may also affect the blood pressure measurement results.

### SUMMARY

According to an aspect, there is provided a blood pressure monitoring cuff which includes: a band that is wound around a measuring position where blood pressure is measured; an air bag that is provided on a first surface on a measuring position side of the band and whose length in a length direction of the first surface is shorter than an outer circumference of the measuring position; a first spacer and a second spacer that are respectively provided at both ends in the length direction of the air bag; and a fixing portion that is provided on the band and is capable of detachably fixing the band, which has been wound around the measuring position so as to cover the first spacer and the second spacer, to a second surface that is an opposite side to the first surface.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 depicts an example configuration of a blood pressure monitor;

FIG. 2 depicts one example of a blood pressure monitor that uses a cuff according to a first embodiment;

FIG. 3 depicts one example of the cuff according to the first embodiment;

FIGS. 4A and 4B depict examples of a spacer provided in the cuff according to the first embodiment;

FIGS. 5A and 5B depict an example of the cuff according to the first embodiment;

FIGS. 6A and 6B depict a cuff that is a comparative example;

FIGS. 7A and 7B depict an example of a cuff according to a second embodiment;

FIGS. 8A to 8C depict how the cuff according to the second embodiment is wound;

FIGS. 9A and 9B depict an example of a cuff according to a third embodiment; and

FIG. 10 depicts an example of a cuff according to a fourth embodiment.

### DESCRIPTION OF EMBODIMENTS

First, a blood pressure monitor will be described.

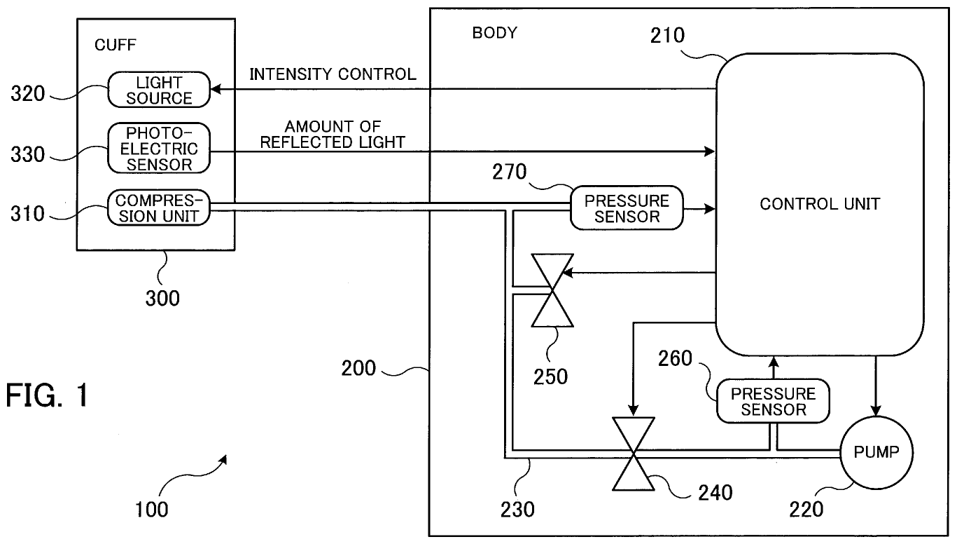

FIG. 1 depicts an example configuration of a blood pressure monitor.

The blood pressure monitor 100 depicted in FIG. 1 includes a body 200 and a cuff 300 that is connected to the body 200.

The body 200 includes a control unit 210, a pump 220, and an air flow path 230 that leads from the pump 220 to the cuff 300. The body 200 further includes a valve 240, a valve 250, a pressure sensor 260, and a pressure sensor 270 provided on the air flow path 230.

The cuff 300 includes a compression unit 310 that compresses the measuring position where blood pressure is measured, and a light source 320 and a photoelectric sensor 330 for detecting the state of an artery (for example, the volume of an artery) at the measuring position. The compression unit 310 is connected to the air flow path 230 and is pressurized and depressurized by introducing air from the air flow path 230 and discharging air to the air flow path 230.

In the blood pressure monitor 100, the pump 220 of the body 200 supplies compressed air to the air flow path 230. The pressure sensor 260 detects the pressure inside the air flow path 230 between the pump 220 and the valve 240. Based on the pressure detected by the pressure sensor 260, the control unit 210 controls the supplying of air from the pump 220 and the opening of the valve 240 so that the pressure (or “pumping pressure”) inside the air flow path 230 between the pump 220 and the valve 240 is constant.

The pressure sensor 270 detects the pressure inside the air flow path 230 between the valve 240 and the compression unit 310 of the cuff 300. The valve 250 is provided on the air flow path 230 between the valve 240 and the compression unit 310 and by opening and closing the valve 250, the air flow path 230 and the external environment are connected and disconnected. In the cuff 300, the light source 320 emits light (for example, infrared light) onto an artery at the measuring position, and the photoelectric sensor 330 detects the amount of reflected light produced for the light emitted by the light source 320 and generates an electric signal indicating the amount of reflected light. The control unit 210 of the body 200 controls the light intensity of the light source 320 and controls the opening of the valve 250 based on the amount of reflected light (i.e., the electrical signal indicating the amount) detected by the photoelectric sensor 330.

The amount of reflected light detected by the photoelectric sensor 330 varies according to the amount of hemoglobin flowing in the artery at the measuring position, and the amount of hemoglobin flowing in the artery varies with the volume of the artery. The control unit 210 controls the opening of the valve 250 and adjusts the pressure of the compression unit 310 so that the reflected light amount detected by the photoelectric sensor 330 is constant. That is, the artery at the measuring position that expands and contracts with pulsation of the heart is compressed by the compression unit 310 so that the volume of the artery is constant. The control unit 210 controls compression of the artery at the measuring position in this way and detects the pressure (cuff pressure) detected by the pressure sensor 270 as the blood pressure at the measuring position.

The blood pressure monitor 100 is a blood pressure measuring apparatus that measures blood pressure of an artery at the measuring position using so-called “volume compensation”, and is capable of noninvasive measurement of blood pressure which may be performed continuously.

A cuff of a configuration described in the following embodiments is used as the cuff 300 of the blood pressure monitor 100 described above.

A first embodiment will be described first.

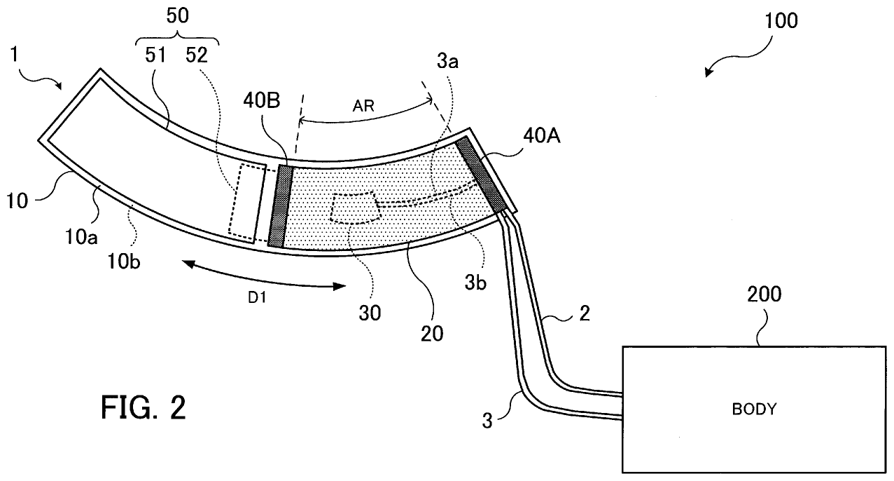

FIG. 2 depicts one example of a blood pressure monitor that uses a cuff according to a first embodiment. FIG. 2 is a plan view schematically depicting the principal part of the cuff.

As one example, assume here that the measuring position for blood pressure is the finger. As depicted in FIG. 2, the blood pressure monitor 100 includes the body 200 and a cuff 1 (corresponding to the cuff 300 (see FIG. 1) described above) connected to the body 200. When measuring the blood pressure at the finger using the blood pressure monitor 100, the cuff 1 is wound around the finger used as the measuring position.

The cuff 1 depicted in FIG. 2 includes a band 10, an air bag 20, a sensor unit 30, a spacer 40A and a spacer 40B, and a fixing portion 50.

When measuring the blood pressure at a finger, the band 10 is wound around the finger. As one example, to match the typical tapered shape of a finger when wound, the band 10 may be curved in the manner of a bow when viewed from above as in FIG. 2.

The air bag 20 is provided on a surface 10a of the band 10 that is located on the finger side when the band 10 is wound around the finger. As depicted in FIG. 2, the air bag 20 is provided in a region AR that starts at a first end in the length direction D1 of the band 10 and is shorter than the outer circumference of the finger at the measuring position. Note that in the example in FIG. 2, in keeping with the band 10 that is curved into a bow shape when viewed from above, an air bag 20 that is shaped like a bow when viewed from above is provided on the surface 10a of the band 10. The air bag 20 expands when pressurized by the air supplied from an air tube 2 and contracts when depressurized by the air discharged to the air tube 2. When blood pressure is measured at the finger, the finger is compressed by the expansion and contraction of the air bag 20. That is, the air bag 20 functions as the compression unit 310 (see FIG. 1) described above.

The sensor unit 30 includes a light source and a photoelectric sensor. As one example, a light-emitting element is used as the light source and a light-receiving element is used as the photoelectric sensor. The sensor unit 30 is a so-called “board-mounted sensor” where a light source and a photoelectric sensor are mounted on a board. The light source of the sensor unit 30 functions as the light source 320 described above (see FIG. 1) and the photoelectric sensor of the sensor unit 30 functions as the photoelectric sensor 330 described above (see FIG. 1). The sensor unit 30 is provided on the inside of the air bag 20, as one example, on the inner surface of the air bag 20.

The spacers 40A and 40B are provided at the respective ends in the length direction D1 of the air bag 20. Since the air bag 20 is provided in the region AR that is shorter than the outer circumference of the finger, the spacers 40A and 40B provided at both ends of the air bag 20 become separated from each other when the band 10 is wound around the finger. The air tube 2 and an electric cable 3 that connect to the body 200 of the blood pressure monitor 100 are connected to one out of the spacer 40A and the spacer 40B, in this example, the spacer 40A. The air tube 2 corresponds to all or part of the air flow path 230 described above (see FIG. 1). The electric cable 3 includes wiring 3a that transmits a signal for controlling intensity to the light source of the sensor unit 30 and wiring 3b that transmits a signal, which has been detected (generated) by the photoelectric sensor of the sensor unit 30, to the body 200. The configuration of the spacer 40A (and the spacer 40B) is described in more detail later.

When the band 10 has been wound onto the finger so as to cover the spacer 40A and the spacer 40B, the fixing portion 50 detachably fixes a second end in the length direction D1, which is opposite the first end where the air bag 20 is provided, to an opposite surface 10b to the surface 10a where the air bag 20 is provided. As one example, a planar fastener 51 and a planar fastener 52 are respectively provided on the surface 10a and the surface 10b of the band 10 as the fixing portion 50. The planar fasteners 51 and 52 are respectively provided at a part of the surface 10a and a part of the surface 10b that face each other when the band 10 has been wound around the finger to cover the spacers 40A and 40B.

The cuff 1 described above is described in more detail below.

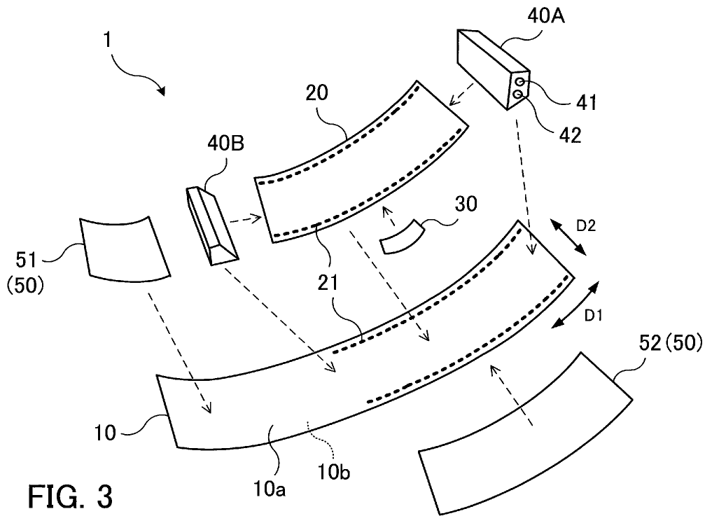

FIG. 3 depicts one example of a cuff according to the first embodiment. FIG. 3 is an exploded perspective view schematically depicting the cuff.

A flexible but non-stretchable or relatively non-stretchable (hereinafter simply “non-stretchable”) sheet, as one example, a non-stretchable vinyl sheet, is used as the band 10 of the cuff 1. A flexible and stretchable or relatively stretchable (hereinafter simply “stretchable”) sheet, as one example, a stretchable vinyl sheet, is stuck on by thermal bonding or the like to the first end in the length direction D1 of the band 10. As one example, as depicted in FIG. 3, thermal bonding is performed at a location 21 to stick the two sheets together. The air bag 20 is formed by the sheet stuck onto the first end of the sheet of the band 10.

The sensor unit 30 is provided inside the air bag 20. As one example, the sensor unit 30 is attached to the inner surface of the sheet of the air bag 20 which is itself stuck onto the sheet of the band 10. The sensor unit 30 is provided inside the air bag 20 so that when the band 10 has been wound with the air bag 20 facing the finger, light from the light source is emitted onto the finger and the reflected light is received by the photoelectric sensor. A stretchable material that is transparent for the light emitted from the light source and the reflected light is used as the sheet of the air bag 20. Various non-stretchable materials are used as the sheet of the band 10, and as one example, a black non-stretchable material is used.

The spacer 40A and the spacer 40B are provided at the respective ends in the length direction D1 of the air bag 20. Various materials are used for the spacer 40A and the spacer 40B to ensure that a certain shape (here meaning a height from the finger) is maintained when the band 10 has been wound around the finger so as to cover the spacers 40A and 40B and the spacers 40A and 40B are themselves pressed down onto the finger by the band 10. As one example, various materials such as resin, cloth, paper, metal, and ceramic are used as the spacers 40A and 40B. The spacers 40A and 40B are attached to the air bag 20 and the band 10 using methods such as thermal bonding or adhesion using adhesive or adhesive tape.

The planar fastener 51 (as one example, loops) and the planar fastener 52 (as one example, hooks) are respectively provided at parts of the air bag 20 side surface 10a and the opposite surface 10b that face each other when the band 10 has been wound around the finger. The planar fasteners 51 and 52 are attached to the band by a method such as adhesion, thermal bonding, or sewing.

The spacers 40A and 40B provided on the cuff 1 will now be described.

The air tube 2, which supplies and discharges air to and from the air bag 20, and the electric cable 3, which includes the wiring 3a and the wiring 3b connected to the sensor unit 30 inside the air bag 20, are provided on one of the spacer 40A and the spacer 40B, in this example, the spacer 40A.

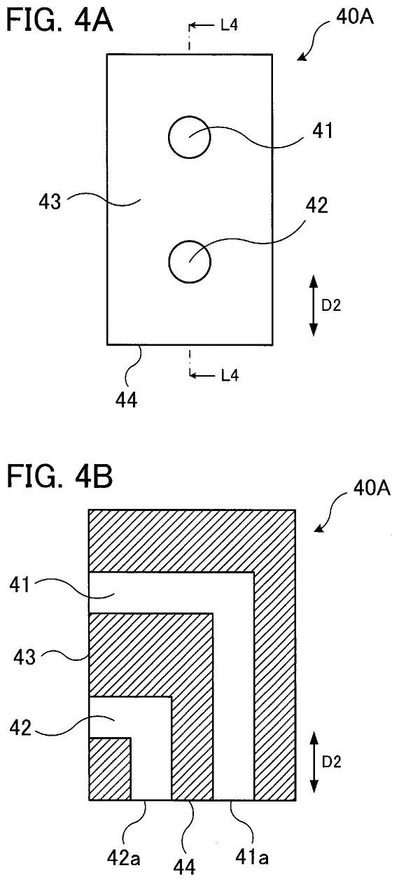

FIGS. 4A and 4B depict examples of a spacer provided in the cuff according to the first embodiment. FIG. 4A is a plan view schematically depicting a principal part when viewed from an attachment surface side of the spacer. FIG. 4B is a cross-sectional view schematically depicting the principal part of the spacer. FIG. 4B is a schematic cross-sectional view along a line L4-L4 in FIG. 4A.

As one example, as depicted in FIGS. 4A, 4B and FIG. 3 described above, the spacer 40A includes a hole 41 that forms the air tube 2 or in which the air tube 2 is provided and a hole 42 in which the electric cable 3 is provided. The spacer 40A is attached so that the hole 41 and the hole 42 pass through to the inside of the air bag 20 provided on the band 10 (“the inside” here meaning the space between the sheet of the band 10 and the sheet of the air bag 20).

The hole 41 and the hole 42 respectively have an opening 41a and an opening 42a that are open to the outside on one side surface 44 at one end in a width direction D2, which is perpendicular to the length direction D1, out of the two surfaces on the sides when an attachment surface 43 where the spacer 40A is attached to the air bag 20 (and the band 10) is regarded as the bottom surface. The hole 41 and the hole 42 extend a certain distance from the opening 41a and the opening 42a in the width distance D2, then bend toward the attachment surface 43, for example by 90° and pass through to the inside of the air bag 20.

Through one of the holes, the hole 41, that passes through to the inside of the air bag 20, air is supplied from the air tube 2, which connects to the body 200, into the air bag 20 and air is discharged from the air bag 20 to the air tube 2. Through the other of the holes, the hole 42, that passes through to the inside of the air bag 20, the electric cable 3 (that is, the wiring 3a and the wiring 3b) is inserted inside the air bag 20 and is connected to the sensor unit 30.

In this way, by providing the air tube 2 that is used to pressurize and depressurize the air bag 20 and the electric cable 3 used to connect the sensor unit 30 on one spacer 40A, it is possible to suppress an increase in the number of the members used for the cuff 1. Also, by providing the air tube 2 and the electric cable 3 on the single spacer 40A, discomfort experienced by the subject when the cuff 1 is wound around the finger is reduced.

Next, the winding of the cuff 1 described above on the finger will be described.

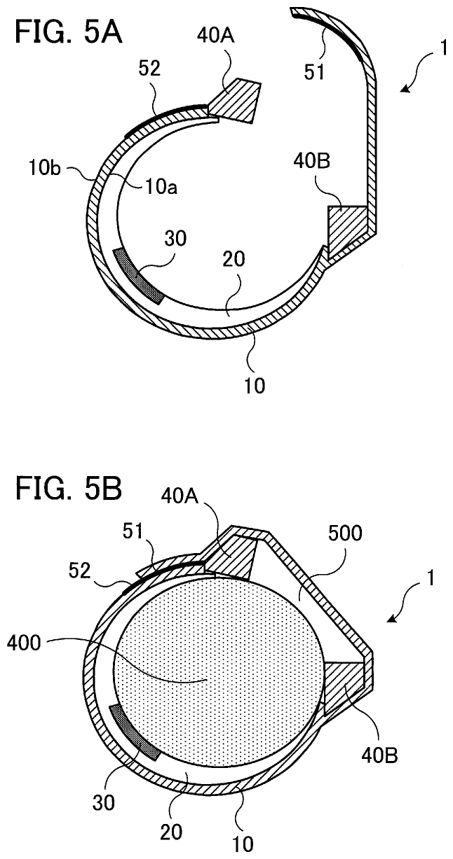

FIGS. 5A and 5B depict an example of a cuff according to the first embodiment. FIG. 5A is a cross-sectional view schematically depicting a principal part of the cuff before winding. FIG. 5B is a cross-sectional view schematically depicting the principal part of the cuff after winding.

As depicted in FIG. 5A, the cuff 1 includes the band 10, the air bag 20 provided on the finger-side surface 10a of the band 10, the sensor unit 30 provided inside the air bag 20, and the spacers 40A and the spacer 40B provided at both ends of the air bag 20. The planar fastener 51 and the planar fastener 52 are respectively provided on the air bag 20 side surface 10a of the band 10 and the opposite surface 10b as the fixing portion 50.

When measuring blood pressure, as depicted in FIG. 5B, the cuff 1 is wound around a finger 400 that is the measuring position for blood pressure. Since the air bag 20 is shorter than the outer circumference of the finger, when the cuff 1 is wound around the finger 400, the spacer 40A and the spacer 40B provided at both ends of the air bag 20 become positioned apart from each other. The finger 400 is therefore exposed between the separated spacers 40A and 40B. The planar fastener 51 end of the band 10 (that is the end where the air bag 20 is not provided) is wound around the finger so as to cover the spacers 40A and 40B that are positioned apart from each other and the planar fastener 51 is fixed to the planar fastener 52 at the facing position. When the band has been fixed in this way, the spacer 40A and the spacer 40B that are positioned apart produce a gap 500 corresponding to the height (thickness) of the spacers 40A and 40B between the finger 400 exposed between the spacers 40A and 40B and the band 10 that has been wound so as to cover the spacers 40A and 40B.

Since the gap 500 is produced between the finger 400 and the band 10 wound around the finger 400, during measurement of blood pressure at (an artery of) the finger 400, even when compression is caused by the air bag 20 expanding due to pressurization, compression of the veins throughout the finger 400 (that is, the position where the cuff 1 is wound) is suppressed. That is, the entire circumference of the finger 400 is not compressed by the cuff 1, producing a state where the part of the finger 400 between the spacers 40A and the spacer 40B is not pressed by the band 10 thanks to the gap 500 and remains unbound. This means that the finger 400 and the veins within the finger are only partially compressed. In this way, with the cuff 1, when measuring the blood pressure at the finger 400, by compressing only some of the veins, congestion of the finger 400, and in particular, congestion during continuous measurement of blood pressure is suppressed.

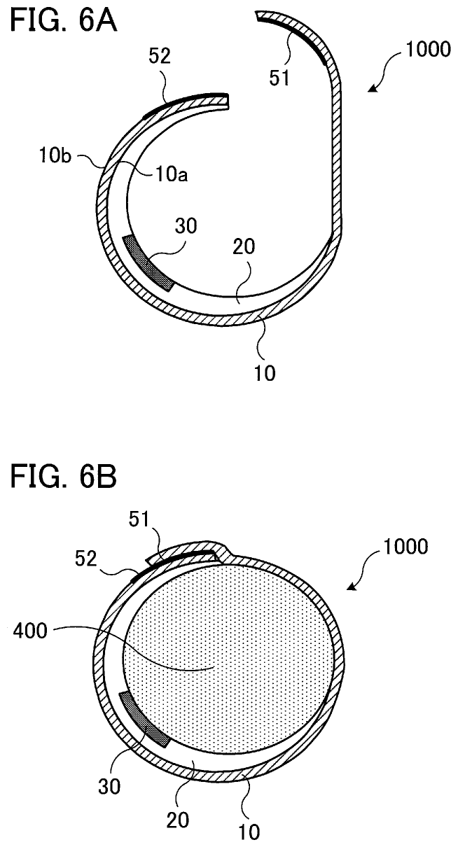

For comparison purposes, an example cuff is depicted in FIGS. 6A and 6B.

FIGS. 6A and 6B are cross-sectional views schematically depicting a principal part of a cuff that is a comparative example respectively before and after attachment.

The cuff 1000 depicted in FIGS. 6A and 6B differs to the cuff 1 described above in that the spacers 40A and 40B are not provided.

When using a configuration, like the cuff 1000, where the spacers 40A and 40B are not provided, the cuff is wound around the finger 400 and the band 10 is fixed using the planar fastener 51 and the planar fastener 52. When the air bag 20 expands, the entire circumference of the finger 400 is squeezed by the cuff 1000. When the entire circumference of the finger 400 is squeezed during measurement of blood pressure in a state where the cuff 1000 is wound around the entire circumference of the finger 400, there is the risk of veins being excessively compressed, causing congestion. Congestion may cause discomfort to the subject and may affect the measurement results for blood pressure.

On the other hand, the cuff 1 described above is provided with the spacers 40A and 40B, and when the cuff 1 is wound around the finger 400 and fixed, part of the finger 400 becomes exposed between the spacers 40A and 40B, with the gap 500 being produced between this part of the finger 400 and the band 10. By doing so, the entire circumference of the finger 400 is not squeezed during measurement of blood pressure and only some of the veins are compressed, which suppresses congestion. Since congestion is suppressed by the cuff 1 in this way, it is possible to reduce the discomfort experienced by the subject and to measure blood pressure accurately at (an artery of) the finger 400.

With the cuff 1, by using flexible sheets as the band 10 and the air bag 20, it is possible to miniaturize the cuff 1 and to reduce the discomfort experienced by the subject when the cuff 1 is wound around the finger 400.

In addition, with the cuff 1, by providing the air tube 2 and the electric cable 3 on one spacer 40A, it is possible to suppress an increase in the number of the members used in the cuff 1 and to reduce the discomfort experienced by the subject when the cuff 1 is wound around the finger 400.

With the cuff 1, the other spacer 40B may be used as a pressing member or a positioning member when the band 10 is wound onto the finger. That is, when the subject himself or herself winds the band 10 onto the finger 400 of one hand, it is possible for example to hold down the spacer 40B with the finger of the other hand and grasp and pull the planar fastener 51 side end of the band 10 with the other fingers and fix the planar fastener 51 to the other planar fastener 52. By using the spacer 40B in this way as a holding member, it is possible to suppress rotation of the cuff 1 in the circumferential direction of the finger 400. By suppressing rotation of the cuff 1, it is possible to guide the cuff 1 so that the sensor unit 30 is located at an appropriate position corresponding to an artery of the finger 400.

According to the first embodiment, the cuff 1, which has superior comfort and attachability on the finger 400, suppresses congestion, and measures the blood pressure of the finger 400 with high accuracy, and the blood pressure monitor 100 that uses this cuff 1 are realized.

A second embodiment will now be described.

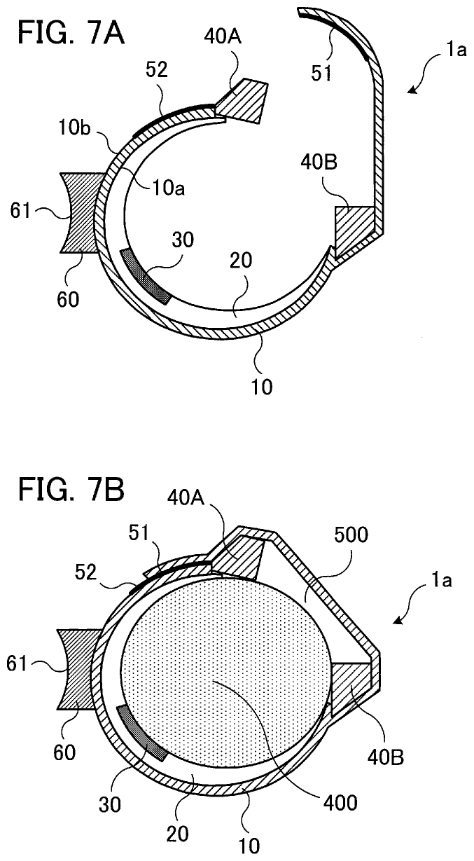

FIGS. 7A and 7B depict a cuff according to the second embodiment. FIG. 7A is a cross-sectional view schematically depicting a principal part of the cuff before attachment and FIG. 7B is a cross-sectional view schematically depicting the principal part of the cuff after attachment.

The cuff 1a depicted in FIGS. 7A and 7B differs to the cuff 1 described above in the first embodiment in that a protruding member 60 is provided on a surface of the band 10 that is located on the outside when the cuff 1a is wound, that is, the surface 10b where the planar fastener 52 is provided.

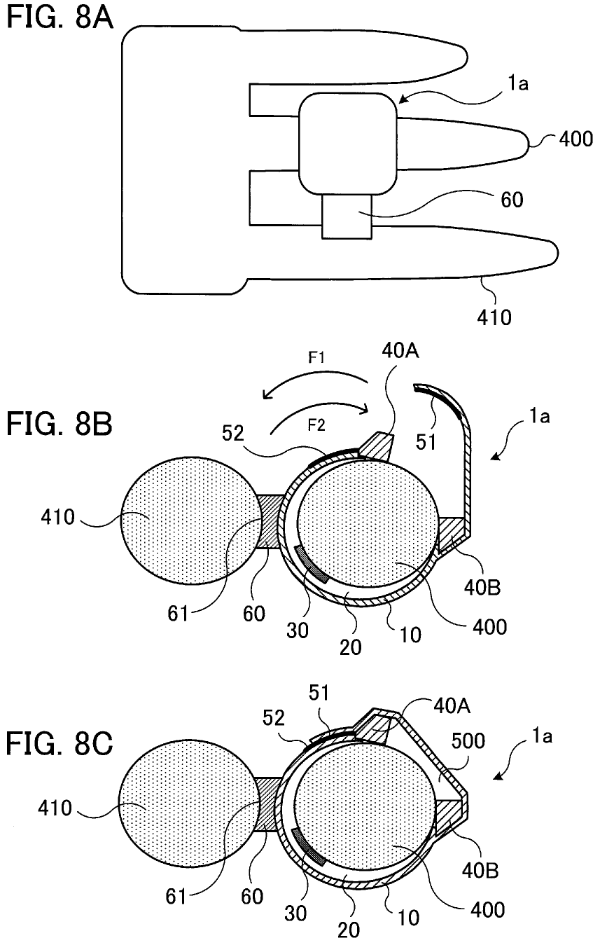

It is possible to use the member 60 of the cuff 1a as a member for holding down or positioning the cuff 1a when the cuff 1a is wound on the finger 400 to be measured as depicted in FIG. 7B. This feature will now be described with reference to FIGS. 8A to 8C.

FIGS. 8A to 8C depict how the cuff according to the second embodiment is wound. FIG. 8A is a plan view schematically depicting a principal part of the hand including the finger where the cuff is to be wound and blood pressure is to be measured. FIGS. 8B and 8C are cross-sectional views schematically depicting a principal part of the hand including the finger where the cuff is to be wound and blood pressure is to be measured. FIG. 8B depicts a state where the cuff is being wound and FIG. 8C depicts a state after the cuff has been wound.

As depicted in FIGS. 8A and 8B, when the cuff 1a where the member 60 is provided on the band 10 is wound onto the finger 400 to be measured, by sandwiching the member 60 between the finger 400 to be measured and an adjacent finger 410, rotation of the cuff 1a is suppressed. In more detail, as depicted in FIG. 8B, during winding of the cuff 1a onto the finger 400 to be measured, when the planar fastener 51 end of the band 10 is pulled and the planar fastener 51 is fixed to the other planar fastener 52, a rotational force F1 in the pulling direction (the circumferential direction of the finger 400) acts upon the cuff 1a. Since the member 60 provided on the cuff 1a has been sandwiched between the finger 400 to be measured and the adjacent finger 410, a rotation resisting force F2 that resists the rotational force F1 is produced, which makes it possible to suppress rotation of the cuff 1a during winding. By doing so, as depicted in FIG. 8C, it is possible to wind the cuff 1a onto the finger 400 to be measured while suppressing rotation of the cuff 1a. By suppressing rotation of the cuff 1a, it is possible to guide the cuff 1a so that the sensor unit 30 becomes located at an appropriate position corresponding to an artery of the finger 400.

So long as the member 60 provided on the cuff 1a is capable of being sandwiched between the finger 400 to be measured and the adjacent finger 410 and suppressing rotation of the cuff 1a, it is possible to use various materials, such as resin, cloth, paper, metal, ceramic, etc. Similarly, the member 60 may be formed in various shapes so long as the member 60 is capable of being sandwiched between the finger 400 to be measured and the adjacent finger 410 and suppressing rotation of the cuff 1a. By providing a depression 61 (see FIGS. 8B and 8C) that is curved so as to fit the external form of the adjacent finger 410 when the member 60 is sandwiched between the fingers and/or providing protrusion that catches on an upper part of the adjacent finger 410, it is possible to suppress rotation of the cuff 1a more effectively.

According to the second embodiment, the cuff 1a, which has superior comfort and attachability on the finger 400, suppresses congestion, and measures the blood pressure of the finger 400 with high accuracy due to the spacers 40A and 40B in combination with the member 60, and the blood pressure monitor 100 that uses the cuff 1a are realized.

A third embodiment will now be described.

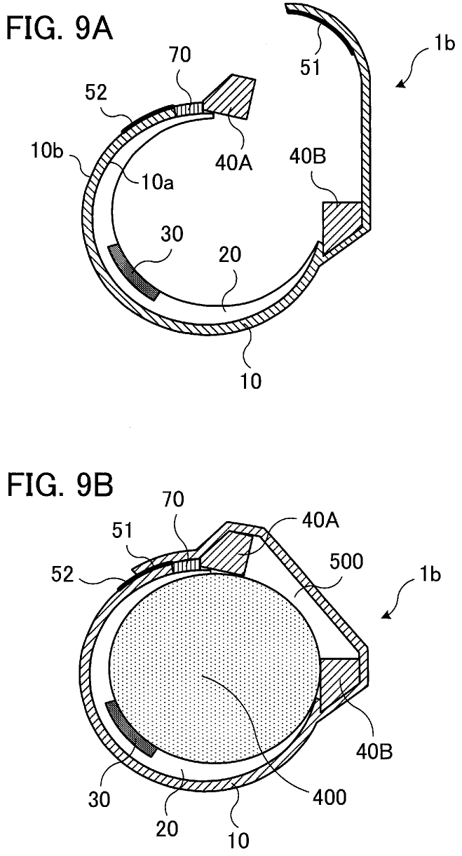

FIGS. 9A and 9B depict one example of a cuff according to the third embodiment. FIG. 9A is a cross-sectional view schematically depicting a principal part of the cuff before winding and FIG. 9B is a cross-sectional view schematically depicting a principal part of the cuff after winding.

A cuff 1b depicted in FIGS. 9A and 9B differs to the cuff 1 according to the first embodiment described above in that a mark 70, which can be identified by the subject or someone else, is provided on a surface of the band 10 that is located on the outside when the cuff 1b is wound, that is, the surface 10b where the planar fastener 52 is provided.

The mark 70 of the cuff 1b is provided at a predetermined position relative to the sensor unit 30. As one example, the mark 70 is provided so that when the cuff 1b has been wound on the finger 400 to be measured and the mark 70 is positioned on the back side of the hand including the finger 400 as depicted in FIG. 9B, the sensor unit 30 becomes located at an appropriate position corresponding to an artery of the finger 400.

When the cuff 1b depicted in FIGS. 9A and 9B is wound onto the finger 400, the planar fastener 51 end of the band 10 is pulled and the planar fastener 51 is fixed to the other planar fastener 52. During this fixing operation or after fixing, by positioning the mark 70 on the back side of the hand with the finger 400, the sensor unit 30 that is at a predetermined position from the mark 70 will become located at an appropriate position corresponding to an artery of the finger 400. By using the mark 70 for guiding so that the sensor unit 30 becomes positioned at an appropriate position corresponding to an artery in the finger 400, it is possible to accurately measure blood pressure at the finger 400.

So long as the mark 70 provided on the cuff 1b can be identified by the subject or someone else, there are no particular limitations on the form of the mark 70. As examples, it is possible to use a mark 70 of a different color to the surface 10b of the band 10, a mark 70 defined by a line on the surface 10b, or a mark 70 that is convex or concave with respect to the surface 10b.

According to the third embodiment, the cuff 1b, which has superior comfort and attachability on the finger 400 due to the spacers 40A and 40B and suppresses congestion and measures blood pressure of the finger 400 with high accuracy due to the spacers 40A and 40B in combination with the mark 70, and the blood pressure monitor 100 that uses the cuff 1b are realized.

Note that the cuff 1b according to the third embodiment may be further equipped with the member 60 described above in the second embodiment. By doing so, it is possible to suppress rotation when the cuff 1b is wound on the finger 400, which makes the cuff 1b easier to attach and increases the accuracy of measurement due to the sensor unit 30 being guided to an appropriate position.

Next, a fourth embodiment will be described.

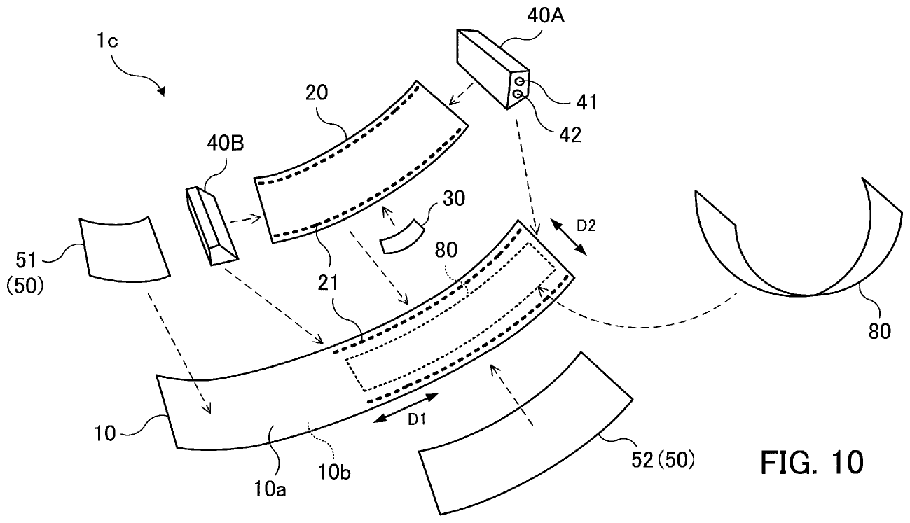

FIG. 10 is an exploded perspective view schematically depicting a cuff according to the fourth embodiment.

The cuff 1c depicted in FIG. 10 differs to the cuff 1 described above in the first embodiment in that a member 80 is provided on the band 10 to maintain the shape of the band 10.

The member 80 uses a sheet that exhibits elasticity, as examples, a variety of resin sheets such as fluorocarbon resin, polyamide resin, and polycarbonate resin. In a solitary state, the member 80 is shaped so as to be curled toward one side. The member 80 is provided on the surface 10b or the surface 10a of the band 10, or as an inner layer of the band 10, so that the curled surface of the member 80 is on the air bag 20 side.

The cuff 1c has the member 80 provided on the band 10 so that before winding on the finger 400, the cuff 1c is curled toward the surface 10a of the band 10, that is, toward the surface 10a on which the air bag 20 is provided. When the cuff 1c is shaped in advance in this way, when the finger is inserted inside the cuff 1c, the cuff 1c will substantially follow the shape of the finger. Since the cuff 1c substantially follows the shape of the finger, a winding operation of pulling the planar fastener 51 end of the band 10 and fixing the planar fastener 51 to the other planar fastener 52 is performed more easily.

According to the fourth embodiment, the cuff 1c, which has superior comfort and attachability on the finger due to the spacers 40A and 40B in combination with the member 80 and is capable of measuring blood pressure at the finger with high accuracy while suppressing congestion due to the spacers 40A and 40B, and the blood pressure monitor 100 that uses the cuff 1c are realized.

Note that the member 60 described above in the second embodiment may be provided on the cuff 1c according to the fourth embodiment. By doing so, it is possible to suppress rotation when the cuff 1c is wound on a finger and possible to make it easier to attach the cuff 1c and measure blood pressure with higher accuracy by guiding the sensor unit 30 to an appropriate position. When the member 60 is provided on the cuff 1c in this way, the member 60 may be connected to the member 80 or may be integrated with the member 80.

The cuff 1c according to the fourth embodiment may also be provided with the mark 70 described above in the third embodiment. By doing so, it is possible to guide the sensor unit 30 to an appropriate position corresponding to an artery in the finger and accurately measure the blood pressure of a finger.

This completes the description of the cuffs 1, 1a, 1b, and 1c according to the first to fourth embodiments that may be used as the cuff 300 of the blood pressure monitor 100. Provided that the spacers 40A and 40B provided in the cuffs 1, 1a, 1b, and 1c produce the gap 500 between the band 10 and the finger, which keeps the band 10 at a fixed height from the finger even when the band 10 is pressed and suppresses congestion, the spacers 40A and 40B may take any shape. As one example, the spacers 40A and 40B are not limited to being trapezoidal in cross section as in the illustrated examples, and it is possible to use spacers that are triangular or semicircular. In addition, protrusions (pressing members) may be additionally provided on the spacers 40A and 40B of this shape to make it easier for the spacers 40A and 40B to grip the finger during winding. The spacers 40A and 40B do not need to have the same shape as each other.

In the example described above, the air tube 2 and the electric cable 3 are provided on a single spacer, the spacer 40A. However, it is also possible to provide the air tube 2 and the electric cable 3 on the other spacer 40B. Alternatively, one spacer, the spacer 40A may be provided with the air tube 2 and the other spacer, the spacer 40B, may be provided with the electric cable 3, or the spacer 40A may be provided with the electric cable 3 and the spacer 40B may be provided with the air tube 2. By providing the air tube 2 and the electric cable 3 on the spacers 40A and 40B, it is possible to suppress an increase in the number of members and decrease discomfort to the subject during winding.

By adjusting the size of the length direction D1 of the band 10 and the air bag 20, it is possible to produce cuffs 1, 1a, 1b, and 1c that match the different outer circumferential sizes of fingers, such as small, medium, and large sizes.

The measuring position of blood pressure is not limited to the finger and may be the wrist, the arm, or the foot. It is possible to realize cuffs of different sizes that have the same configurations as the cuffs 1, 1a, 1b, and 1c and can be wound around different measuring positions of blood pressure.

According to the embodiments, there is realized a blood pressure monitoring cuff that suppresses congestion during measurement of blood pressure.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that various changes, substitutions, and alterations could be made hereto without departing from the spirit and scope of the invention.

What is claimed is:

1. A blood pressure monitoring cuff comprising:
a band that is wound around a measuring position where blood pressure is measured;
an air bag that is provided on a first surface on a measuring position side of the band and whose length in a length direction of the first surface is shorter than an outer circumference of the measuring position;
a first spacer and a second spacer that are respectively provided at both ends in the length direction of the air bag; and
a fixing portion that is provided on the band and is capable of detachably fixing the band, which has been wound around the measuring position so as to cover the first spacer and the second spacer, to a second surface that is an opposite side to the first surface.

2. The blood pressure monitoring cuff according to claim 1,
wherein the first spacer has a first hole, which passes through to an inside of the air bag and through which air that pressurizes and depressurizes the air bag flows.

3. The blood pressure monitoring cuff according to claim 2,
wherein the first hole bends after extending in a width direction of the first surface and passes through to the inside of the air bag.

4. The blood pressure monitoring cuff according to claim 1, further comprising a sensor unit provided inside the air bag,
wherein the first spacer or the second spacer has a second hole that passes through to the inside of the air bag and in which a wire that is connected to the sensor unit is disposed.

5. The blood pressure monitoring cuff according to claim 1, further comprising a first member that is provided on the second surface and is capable of being interposed between the measuring position where the band is wound and a different part of a body of a measured subject to the measuring position.

6. The blood pressure monitoring cuff according to claim 1, further comprising a mark that is provided on the second surface and corresponds to a position that is a predetermined distance in the length direction from the sensor unit.

7. The blood pressure monitoring cuff according to claim 1, further comprising a second member that is provided on the band and holds the band in a certain shape before winding on the measuring position.

8. A blood pressure monitoring cuff comprising:
a band that is wound around a measuring position where blood pressure is measured;
an air bag that is provided on a first surface on a measuring position side of the band; and
a first spacer that is provided at one end of the air bag in a length direction of the first surface and has a first hole that passes through to an inside of the air bag and through which air that pressurizes and depressurizes the air bag flows.

9. A blood pressure monitoring cuff comprising:
a band that is wound around a measuring position where blood pressure is measured;
an air bag that is provided on a first surface on a measuring position side of the band; and
a first member that is provided on a second surface, which is an opposite side to the first surface, and is capable of being interposed between the measuring position where the band is wound and a different part of a body of a measured subject to the measuring position.
